SEQUENCE LISTING (4 sequences)

SEQ ID NO: 1 - protein, 194 aa, Artificial Sequence
  RBD amino acid sequence
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 2 - protein, 163 aa, Artificial Sequence
  HP_Ferritin amino acid sequence
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 3 - protein, 360 aa, Artificial Sequence
  fusion protein RBD-HP_Ferritin amino acid sequence (not containing SP-His-tag)
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 4 - protein, 395 aa, Artificial Sequence
  fusion protein RBD-HP_Ferritin amino acid sequence (containing SP-His-tag)
MGILPSPGMPALLSLVSLLSVLLMGCVAEHHHHHHNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

(19) United States
(12) Patent Application Publication
(10) Pub. No.: US 2023/0173057 A1
(43) Pub. Date: Jun. 8, 2023

(54) METHOD FOR IMPROVING ANTIGEN IMMUNOGENICITY, CORONAVIRUS ANTIGEN, USE THEREOF, RECOMBINANT VECTOR, EXPRESSION KIT, TRANSGENIC CELL LINE, RECOMBINANT BACTERIUM, CORONAVIRUS VACCINE, PREPARATION METHOD OF ANTIGEN AND NUCLEOTIDE SEQUENCE

(71) Applicant: GUANGZHOU QIANYANG BIO-TECHNOLOGY PHARMACEUTICAL CO., LTD., Guangdong (CN)

(72) Inventors: Hui ZHANG, Guangdong (CN); Xiancai MA, Guangdong (CN); Fan ZOU, Guangdong (CN); Yaochang YUAN, Guangdong (CN); Rong LI, Guangdong (CN); Xu ZHANG, Guangdong (CN)

(73) Assignee: GUANGZHOU QIANYANG BIO-TECHNOLOGY PHARMACEUTICAL CO., LTD., Guangdong (CN)

(21) Appl. No.: 17/912,525

(22) PCT Filed: Mar. 30, 2020
(86) PCT No.: PCT/CN2020/082037

§ 371 Date: Sep. 19, 2022

(30) Foreign Application Priority Data
Mar. 20, 2020 (CN) 202010203031.2

(51) Int. Cl. A61K 39/215; C12N 15/86; C07K 14/005; C07K 14/20; A61P 31/14

(52) U.S. Cl. CPC A61K 39/215; A61P 31/14; C07K 14/005; C07K 14/20; C12N 15/86; C07K 2319/02; C07K 2319/21; C12N 2770/20022; C12N 2770/20034; C12N 2770/20043

(57) ABSTRACT

Provided is a Helicobacter pylori ferritin-based novel coronavirus S protein single-region subunit nano-vaccine. According to the present invention, a receptor binding domain (RBD) of a virus is used as an antigen and is connected with a Helicobacter pylori multimeric protein (HP_Ferritin) to form a fusion protein RBD-HP_Ferritin, such that antigen multimerization is realized; and an eukaryotic cell expression system is then utilized for expression, so as to form a 24-mer nano-antigen by means of the self-assembly action of the HP_Ferritin. According to the solution, the defect that RBD monomers are insufficient in immunogenicity can be overcome; the obtained vaccine can remarkably improve the level of neutralizing antibodies of a host to viruses; and the generated antibodies have the capacity to strongly prevent the viruses from invading target cells.

### TECHNICAL FIELD

The invention belongs to the technical field of biomedicine, and more specifically relates to a Helicobacter pylori ferritin-based novel coronavirus (2019-nCoV) S protein single-region subunit nano-vaccine.

### BACKGROUND

Since December 2019, a series of pneumonia cases of unknown cause have occurred in Wuhan Hubei, China, which clinical manifestations are very similar to those of viral pneumonia; the main clinical manifestations are fever, fatigue, dry cough, etc. In severe cases, shock, sepsis, respiratory failure may occur, causing death. Deep sequencing analysis of nine cases of lower respiratory tract samples was utilized to reveal presence of a novel coronavirus, tentatively known as SARS-CoV-2 (also known as 2019-nCoV). As of February 19, more than 70,000 patients have been confirmed in China, and there are still more than 5,000 suspected cases, resulting in more than 1,600 deaths, and hundreds of cases have also been confirmed in Japan, Thailand, South Korea, the United States, and many countries in Europe, having a momentum of spreading in China and even the world. Due to unclear source and pathogenesis of the novel coronavirus pneumonia, and lack of specific antiviral drugs, it has brought great difficulties to clinical diagnosis and treatment and control of the epidemic, resulting in a serious social burden and crisis.

At present, humans still lack an effective vaccine against SARS-CoV-2. Under this severe situation, developing a safe and effective vaccine against SARS-CoV-2 as soon as possible to protect susceptible population is of great significance to public health and national security.

For development of vaccines, the structure of the virus must be understood first. Coronaviruses belong to the class of enveloped single positive-stranded RNA viruses that widely exist in humans and other mammals as well as birds, and cause respiratory, digestive, liver and nervous system diseases. Before this outbreak, six coronaviruses have been known to cause diseases in humans. Among them, four coronaviruses 229E, OC43, NL63 and HKU1 basically only cause common cold symptoms in immunocompromised people, while the other two, well known as SARS-CoV and MERS-CoV, can cause severe infectious diseases. Length of a single-stranded positive RNA genome at a 5′ end of the coronavirus is between 26.2 and 31.7 kb, which is the longest among all RNA viruses. Its genome has six to ten open reading frames (ORF). The first ORF contains two thirds of the genome and encodes and reproduces enzyme proteins, while the last third contains a fixed-order structural protein gene: (HE)-S-E-M-N. There are multiple ORFs encoding accessory proteins between these genes. The genome is packaged into a helical nucleocapsid surrounded by a host-derived lipid bilayer. This viral membrane contains at least three viral proteins, that is, spike protein (S), membrane protein (M) and envelope protein (E).

Among them, M protein and E protein are mainly involved in the assembly of the virus, while S protein mediates the virus to bind to receptors on host cell membrane and fuse with the host cell membrane. Therefore, the S protein plays an important role in tissue tropism, cell fusion and virulence of the virus, and contains main neutralizing epitopes of the coronavirus. A receptor binding domain (RBD) of S protein of MERS-CoV and SARS-CoV is considered to be the most important antigen target region for inducing neutralizing antibodies in body. As a vaccine, RBD can make the neutralizing antibodies produced by stimulation of the body more focus on the receptor binding against the virus, which can improve immunogenicity and immune efficiency of the vaccine. In addition, MERS-CoV invades cells through RBD binding to a host cell receptor (CD26, also known as DPP4), and SARS-CoV enters cells through its RBD binding to a host cell receptor ACE2. As a core of the vaccine, RBD can make the neutralizing antibodies produced by stimulation of the body more focus on the receptor binding against the virus, thereby improving the immunogenicity and neutralization efficiency of the vaccine. However, in previous studies, after vaccination in animal models, RBD monomer vaccine derived from MERS-CoV and SARS-CoV only elicited low levels of pseudotyped virus neutralizing antibodies.

Therefore, it is urgent to develop vaccines with high immunogenicity and neutralization efficiency against coronaviruses, especially SARS-CoV-2.

### SUMMARY

The technical problem to be solved by the present invention is to overcome the deficiencies of existing therapeutic drugs and vaccines against novel coronavirus, and to develop a safe and effective vaccine against SARS-CoV-2 as soon as possible to protect susceptible population. In the present invention, taken receptor binding domain (RBD) of the virus as single antigen fragment, and based on Helicobacter pylori multimeric protein (Helicobacter pylori_Ferritin, Ferritin), an antigen multimerization is realized and an RBD antigen multimeric complex is constructed and developed. Specifically, a receptor binding domain (RBD) of a virus is used as single antigen fragment and is connected with a Helicobacter pylori multimeric protein (Helicobacter pylori_Ferritin, Ferritin(HP)) to form a fusion protein RBD-HP_Ferritin, such that antigen multimerization is realized. At the same time, a signal peptide and a purification tag are added, and a self-assembled RBD-HP_Ferritin protein is expressed through a plasmid transfection eukaryotic cell expression system (such as 293F cells), RBD-HP_Ferritin monomers can be assembled into a spherical 24-mer nanoparticle through self-assembly of Ferritin(HP), displayed on surface of nanoparticle, which overcomes shortcomings of insufficient immunogenicity of RBD monomers, and can effectively cause a stronger immune response and produce antibodies neutralizing SARS-CoV-2 pseudotyped virus invading target cells. The vaccine of the present invention can significantly improve a neutralizing antibody level of the host against SARS-CoV-2; and the vaccine preparation method of the present invention is simple, the protein contains a His tag and is easy to purify, safety of Ferritin antigen as a carrier of nano-vaccine has been proved in clinical trials registered by NIH, and the vaccine can be applied to clinical trials more quickly.

An objective of the present invention is to provide a method for improving antigen immunogenicity.

Another objective of the present invention is to provide a 24-multimerized subunit novel coronavirus antigen constructed based on a receptor binding region of novel coronavirus (SARS-CoV-2) and a bacterial multimer.

Another objective of the present invention is to provide an application of the novel coronavirus antigen in preparation of novel coronavirus vaccine and anti-novel coronavirus medicament.

Another objective of the present invention is to provide a method for preparing the novel coronavirus antigen.

Another objective of the present invention is to provide a nucleotide sequence, a vector or a transgenic cell line that encodes and expresses the novel coronavirus antigen.

The above-mentioned objectives of the present invention are achieved through the following technical solutions.

The present invention first provides a method for improving antigen immunogenicity, the method is to combine the receptor binding domain (RBD) of the virus with Helicobacter pylori multimeric protein (Helicobacter pylori_Ferritin, Ferritin (HP)) to form a new fusion protein RBD-HP_Ferritin, as an antigen.

As a self-assembled globular protein, Ferritin has an amino terminal spacing of about 4.5-7.5 nm for every two adjacent subunits on its surface, which is suitable for loading antigens on an outer surface. Using such a characteristic that HP_Ferritin, a ferritin derived from Helicobacter pylori, enables to spontaneously form multimerization, and after the surface is loaded with antigens, it can induce strong humoral immune response and cellular immune response, it is a very ideal carrier, and can increase the number of antigens that can be carried by a single immunization.

In the solution for improving antigen immunogenicity of the present invention, taking receptor binding domain (RBD) of the virus as antigen fragments, and based on Helicobacter pylori multimeric protein (Helicobacter pylori_Ferritin, Ferritin), an antigen multimerization is realized, which can overcome shortcomings of insufficient immunogenicity of RBD monomers, can effectively cause a stronger immune response, and can significantly improve the level of neutralizing antibodies of a host against SARS-CoV-2.

In the past antigen research, especially the SARS research, only immunogenicity of a certain segment, e.g. RBD region is focused, but the current research and development of related vaccines have all failed. We carried out multimerization of HP_Ferritin on the antigen fragments. Using a characteristic that HP_Ferritin (a ferritin derived from Helicobacter pylori) can spontaneously form multimerization, antigens are aggregated together to form a nanoparticle, which further increase the number of antigens carried in a single immunization, so it can more fully and stably contact immune cells in the human body to stimulate the production of antibodies. The “multimer” strategy of the present invention can achieve the effect of stimulating the body to produce an effective immune response more effectively, rapidly and stably in terms of quantity (multimerization).

Preferably, the above antigen of the present invention is preferably suitable for a coronavirus antigen, and the receptor binding domain RBD of the virus are a receptor binding domain RBD of a coronavirus.

Preferably, a novel coronavirus SARS-CoV-2 antigen is included, and the receptor binding domain RBD of the coronavirus are a receptor binding domain RBD of a novel coronavirus SARS-CoV-2.

More specifically, preferably it means that the novel coronavirus SARS-CoV-2 antigen is a surface spike protein (S protein) neutralizing antigen of novel coronavirus SARS-CoV-2, the receptor binding domain RBD of the coronavirus are a receptor binding domain RBD of a novel coronavirus SARS-CoV-2.

Specifically, an amino acid sequence of the RBD of the novel coronavirus SARS-CoV-2 is shown in SEQ ID NO: 1.

An amino acid sequence of the Ferritin(HP) is shown in SEQ ID NO: 2.

SEQ ID NO: 1 and SEQ ID NO: 2 can be directly linked to obtain a new fusion protein.

Alternatively, SEQ ID NO: 1 and SEQ ID NO: 2 are linked by a hinge region Linker to form a new fusion protein RBD-HP_Ferritin. As an alternative preferred solution, the Linker may be GSG. When the Linker is GSG, an amino acid sequence of the resulting fusion protein RBD-HP_Ferritin is shown in SEQ ID NO: 3.

Further preferably, as an alternative embodiment, the method for improving antigen immunogenicity described in the present invention is to combine receptor binding domain (RBD) of a virus with Helicobacter pylori multimeric protein (Helicobacter pylori_Ferritin, Ferritin(HP)) to form a fusion protein RBD-FP-HP_Ferritin, then add a signal peptide and a purification tag, and express an antigen through an eukaryotic expression system.

Preferably, the signal peptide is a secretory signal peptide (SP). Preferably, the purification tag is a His tag (His-tag). The signal peptide and the purification tag are added to an amino acid N-terminal of the RBD.

After adding the signal peptide and the purification tag, an amino acid sequence of a fusion protein RBD-HP_Ferritin obtained by fusion of SP, His-tag, RBD of novel coronavirus SARS-CoV-2, and Ferritin (HP) is shown in SEQ ID NO: 4 (as shown in FIG. 2).

That is, the present invention provides a SARS-CoV-2 antigen with improved immunogenicity containing a signal peptide and a purification tag, the antigen is a protein RBD-HP_Ferritin using Helicobacter pylori ferritin to self-assemble proteins and to be 24-multimerized (as shown in FIG. 1).

The Helicobacter pylori multimeric protein (Helicobacter pylori_Ferritin, Ferritin (HP)) is a bacterial complex ferritin, and the bacterial complex ferritin forms a spherical protein presenting in bacterium, which primarily acts to control a rate and location of polynuclear ferric oxide formation, via transport of hydrated iron ion and proton to and from a mineralized core. A globular form of ferritin is composed of a monomeric subunit protein (Ferritin), which is a polypeptide with a molecular weight of about 17-20 kD. The sequence of one such monomeric ferritin subunit is shown in SEQ ID NO: 2. These monomeric ferritin subunit proteins self-assemble into a globular ferritin protein containing 24 monomeric ferritin subunit proteins.

The fusion protein RBD-HP _Ferritin can assemble RBD-HP _Ferritin monomers into a spherical 24-mer nanoparticle through self-assembly of Ferritin (HP), displaying RBDs on surface of the nanoparticle, which can effectively elicit a stronger immune response from the receptor, producing antibodies that neutralize SARS-CoV-2 pseudotyped virus invading target cell. The 24-multimerized RBD-HP_Ferritin of the present invention can overcome shortcoming of insufficient immunogenicity of RBD monomer, and significantly improve production of neutralizing antibodies of the receptor against SARS-CoV-2.

The present invention also provides a coronavirus antigen with improved immunogenicity, specifically a new self-assembled and 24-multimerized fusion protein RBD-HP_Ferritin constructed by the above method.

The amino acid sequence of the novel coronavirus SARS-CoV-2 antigen (a new fusion protein RBD-HP_Ferritin) is shown in SEQ ID NO: 3 (constructed by linking SEQ ID NO: 1 and SEQ ID NO: 2 by a hinge region GSG); or the amino acid sequence formed by adding a signal peptide and a purification tag is shown in SEQ ID NO: 4.

That is, as an alternative preferred embodiment of the present invention, the novel coronavirus SARS-CoV-2 antigen (a new fusion protein RBD-HP_Ferritin) contains a signal peptide and a purification tag disclosed herein, RBD protein, and self-assembled subunit protein Ferritin are linked, wherein the RBD-HP_Ferritin protein can self-assembly into a nanoparticle that displays an immunogenic portion of the RBD protein on its surface. After further safety and efficacy studies in animal models, the RBD-HP_Ferritin vaccine has a potential to protect SARS-CoV susceptible population.

Therefore, an application of the coronavirus antigen in preparation of anti-coronavirus medicaments, specifically including an application in preparation of medicaments against novel coronavirus SARS-CoV-2, which is also within protection scope of the present invention.

As an alternative embodiment, an anti-SARS-CoV-2 vaccine can be prepared by using RBD-HP_Ferritin protein in combination with a SAS adjuvant.

In addition, as an alternative embodiment, the application also includes an application in preparation of a kit; the kit contains the protein antigen, or a DNA molecule encoding the antigen, or a recombinant vector/ expression kit/ transgenic cell line/ recombinant bacterium expressing the antigen.

In addition, the present invention further provides a recombinant vector, expression kit, transgenic cell line or recombinant bacterium expressing the above-mentioned antigen (fusion protein RBD-FP-HP_Ferritin).

Finally, the present invention further provides an alternative preparation method of the above antigen, specifically, at a 3′ end of a nucleotide sequence corresponding to amino acids as shown in direct linking or hinge linking of SEQ ID NO: 1 and SEQ ID NO: 2, or a nucleotide sequence corresponding to amino acids as shown in SEQ ID NO: 3, or a nucleotide sequence corresponding to amino acids as shown in SEQ ID NO: 4, adding a translation terminator codon, cloned into an eukaryotic expression vector (as shown in FIG. 3, pcDNA3.1-Intron-WPRE), after enzyme cleavage and correct sequencing (as shown in FIG. 4), expressing an nano-antigen in a transient transfection eukaryotic expression system (e.g. 293F cell) (as shown in FIG. 5), collecting a cell supernatant after expression, and purifying to obtain the novel coronavirus SARS-CoV-2 antigen (a multimeric RBD protein).

As an alternative embodiment, the eukaryotic expression system includes, but is not limited to, HEK293T cell, 293F cell, CHO cell, sf9 and other cell strains and cell lines that can be used to express eukaryotic proteins. Protocols for introducing corresponding proteins into the eukaryotic expression system include, but are not limited to, transfection, infection, transposition protocols, and the like.

As an alternative embodiment, the purification method is filtering the supernatant of cells expressing the antigen to remove cell debris, and then passing through a 10 K ultrafiltration tube (Millipore) for preliminary purification, and then passing through a HisTrap HP nickel column (GE), Lectin column (GE) to capture the target protein, and finally purifying by molecular sieve chromatography using Siperose6 Increase 10/300 GL column (GE) to obtain a high-purity target protein (as shown in FIGS. 6-7).

As an alternative embodiment, a buffer for ultrafiltration elution is: PBS buffer at pH 7.4.

As an alternative embodiment, a buffer for nickel column elution is: PBS at pH 7.4, containing 500 mM Imidazole.

As an alternative embodiment, a packing material of Lectin column (GE) is: Concanavalin A (Con A), Wheat germ agglutinin (WGA), and an eluent for column elution is: methyl-α-D-mannopyranoside, GlcNAc.

As an alternative embodiment, a buffer for molecular sieve chromatography is: PBS buffer at pH 7.4.

The nano-vaccine obtained in the present invention is a purified 24-multimerized RBD-HP _Ferritin protein; size of the 24-multimerized RBD-HP _Ferritin protein is about 46 Kd under a non-reducing condition (without DTT added).

Finally, a nucleotide sequence encoding and expressing the above-mentioned antigen of the present invention, as well as a vectors or a transgenic cell line containing the nucleotide sequence, encoding and expressing the antigen, shall also fall within the protection scope of the present invention.

The present invention relates to the sequence as follows:

SEQ ID NO: 1 (amino acid sequence of RBD)
1 NITNLCPFGE VFNATRFASV YAWNRKRISN CVADYSVLYN SASFSTFKCY
GVSPTKLNDL
61 CFTNVYADSF VIRGDEVRQI APGQTGKIAD YNYKLPDDFT GCVIAWNSNN
LDSKVGGNYN
121 YLYRLFRKSN LKPFERDIST EIYQAGSTPC NGVEGFNCYF PLQSYGFQPT
NGVGYQPYRV
181 VVLSFELLHA PATV

SEQ ID NO: 2 (amino acid sequence of HP_Ferritin)
1 DIIKLLNEQV NKEMQSSNLY MSMSSWCYTH SLDGAGLLFLF DHAAEEYEHA
KKLIIFLNEN
61 NVPVQLTSIS APEHKFEGLT QIFQKAYEHE QHISESINNI VDHAIKSKDH
ATFNFLQWYV
121 AEQHEEEVLF KDILDKIELI GNENHGLYLA DQYVKGIAKS RKS

SEQ ID NO: 3 (amino acid sequence of fusion protein RBD-HP_Ferritin, not containing
SP-His-tag)
1 NITNLCPFGE VFNATRFASV YAWNRKRISN CVADYSVLYN SASFSTFKCY
GVSPTKLNDL
61 CFTNVYADSF VIRGDEVRQI APGQTGKIAD YNYKLPDDFT GCVIAWNSNN
LDSKVGGNYN
121 YLYRLFRKSN LKPFERDIST EIYQAGSTPC NGVEGFNCYF PLQSYGFQPT
NGVGYQPYRV
181 VVLSFELLHA PATVGSGDII KLLNEQVNKE MQSSNLYMSM SSWCYTHSLD
GAGLFLFDHA
241 AEEYEHAKKL IIFLNENNVP VQLTSISAPE HKFEGLTQIF QKAYEHEQHI
SESINNIVDH
301 AIKSKDHATF NFLQWYVAEQ HEEEVLFKDI LDKIELIGNE NHGLYLADQY
VKGIAKSRKS

SEQ ID NO: 4 (amino acid sequence of fusion protein RBD-HP_Ferritin, containing
SP-His-tag)
1 MGILPSPGMP ALLSLVSLLS VLLMGCVAEH HHHHHNITNL CPFGEVFNAT
RFASVYAWNR
61 KRISNCVADY SVLYNSASFS TFKCYGVSPT KLNDLCFTNV YADSFVIRGD
EVRQIAPGQT
121 GKIADYNYKL PDDFTGCVIA WNSNNLDSKV GGNYNYLYRL FRKSNLKPFE
RDISTEIYQA
181 GSTPCNGVEG FNCYFPLQSY GFQPTNGVGY QPYRVVVLSF ELLHAPATVG
SGDIIKLLNE
241 QVNKEMQSSN LYMSMSSWCY THSLDGAGLF LFDHAAEEYE HAKKLIIFLN
ENNVPVQLTS
301 ISAPEHKFEG LTQIFQKAYE HEQHISESIN NIVDHAIKSK DHATFNFLQW
YVAEQHEEEV
361 LFKDILDKIE LIGNENHGLY LADQYVKGIA KSRKS

The present invention has the following beneficial effects.

In the present invention, a receptor binding domain (RBD) of a virus is used as an antigen fragment, and combined with Helicobacter pylori multimeric protein (Helicobacter pylori_Ferritin, Ferritin (HP)) to form a fusion protein RBD-HP_Ferritin, such that antigen multimerization is realized, at the same time, a signal peptide and a purification tag are added, and a self-assembled RBD-HP_Ferritin protein is expressed through a plasmid transfection eukaryotic cell expression system (such as 293F cells), RBD can be assembled into a 24-multimerized nano-vaccine through self-assembly of HP_Ferritin. This solution can overcome shortcomings of insufficient immunogenicity of RBD monomers, and the resulting vaccine can significantly increase a level of neutralizing antibodies against SARS-CoV-2 in the host. In the present invention, the experiment of immunizing Balb/c mice with RBD-HP_Ferritin nano-antigen has confirmed that the antibody produced has an ability to strongly block SARS-CoV-2 pseudotyped virus from invading target cells.

In addition, the vaccine preparation method of the invention is simple, the protein contains a His tag and is easy to purify, and the safety of Ferritin antigen as a carrier of nano-vaccine has been proved in clinical trials registered by NIH, and the vaccine can be quickly applied to clinical trials.

### DESCRIPTION OF DRAWINGS

FIG. 1 is a schematic diagram of self-assembly of RBD-HP_Ferritin fusion proteins into a nanoparticle.

FIG. 2 is a schematic diagram of structure of the RBD-HP_Ferritin fusion protein.

FIG. 3 is a schematic diagram of structure of plasmid expressing RBD-HP_Ferritin.

FIG. 4 is an enzyme cleavage verification of RBD-HP_Ferritin fusion.

FIG. 5 is an immunofluorescence image of 293F cells transfected with RBD-HP_Ferritin fusion protein.

FIG. 6 is a molecular sieve diagram for purification of RBD-HP_Ferritin fusion protein.

FIG. 7 is a SDS-PAGE image for purification of RBD-HP_Ferritin fusion protein (about 48 KD).

FIG. 8 is an immunization strategy for mice immunized with RBD-HP _Ferritin nano-vaccine.

FIG. 9 is a detection strategy for neutralizing antibody titer in mouse serum.

FIG. 10 shows that mice immunized with RBD-HP _Ferritin nano-vaccine produce neutralizing antibodies that block SARS-CoV-2 from invading into target cells.

FIG. 11 shows experimental results of the fusion protein RBD-PF_Ferritin in Comparative Example 1.

FIG. 12 shows experimental results of the fusion protein LS-RBD in Comparative Example 2.

### DETAILED DESCRIPTION

The present invention is further described below with reference to the accompanying drawings and specific embodiments, but the embodiments do not limit the present invention in any form.

Unless otherwise specified, reagents, methods and equipment used in the present invention are conventional reagents, methods and equipment in the technical field.

Unless otherwise specified, the reagents and materials used in the following embodiments are commercially available.

### Embodiment 1 Construction of Novel Coronavirus SARS-CoV-2 Antigen (Fusion Protein RBD-HP _Ferritin)

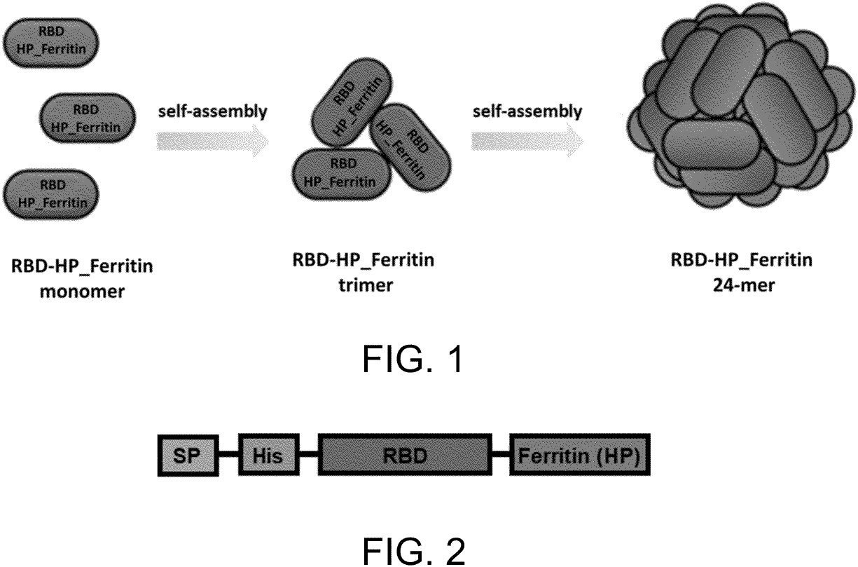

The schematic diagram of self-assembly of RBD-HP_Ferritin fusion proteins into a nanoparticle, and the schematic diagram of structure is as shown in FIG. 1 and FIG. 2, respectively..

Specifically, construction and preparation method of fusion protein RBD-HP_Ferritin is as follows:

### 1. Preparation of Vector Expressing RBD-Ferritin Antigen

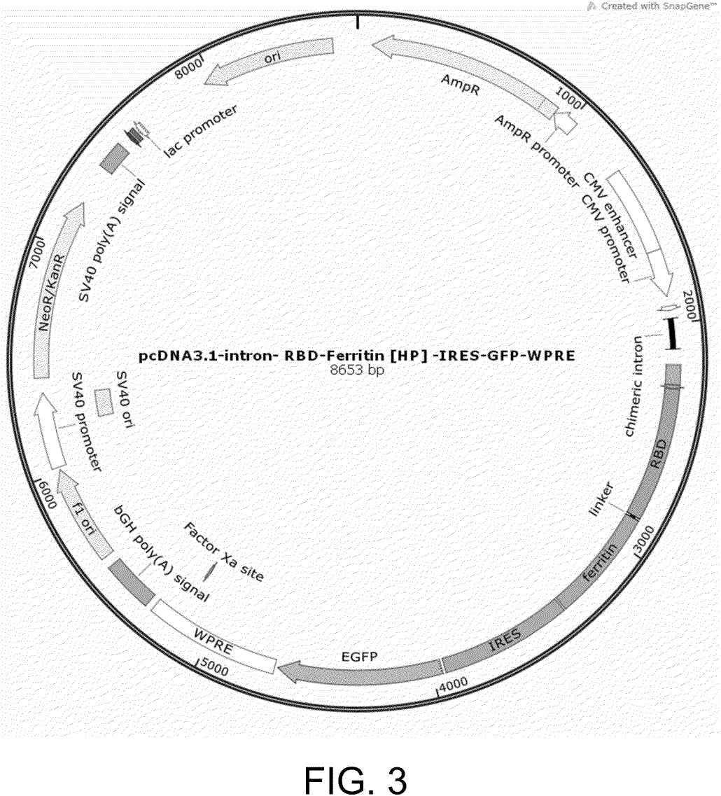

A translation terminator codon was added at a 3′ end of a nucleotide sequence of RBD-HP_Ferritin, which was then cloned and added between Xho I and Xba I enzyme cleavage sites in an Intron and WPRE expression-enhanced expression vector (pcDNA3.1-Intron-WPRE), and an expression vector pcDNA3.1-Intron-WPRE-RBD-Ferritin(HP)-IRES-GFP (as shown in FIG. 3) was constructed.

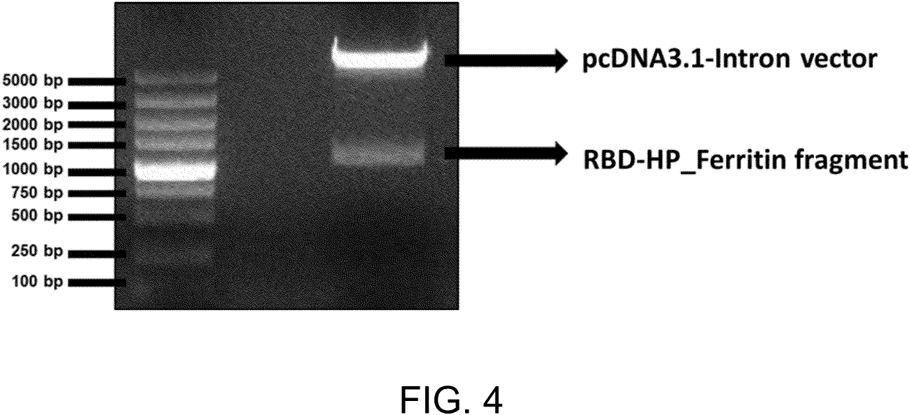

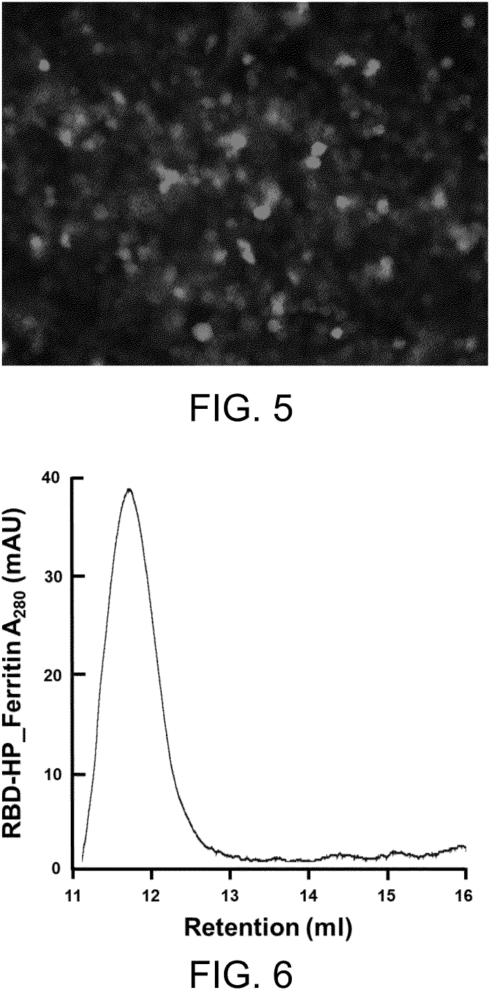

The recombinant plasmid was transformed into DH5α competent cells, cultured at 37° C. overnight, and positive clones were screened and identified by PCR. An endotoxin-depleted plasmid was extracted, then after enzyme cleavage and verification by sequencing, it was used for expression of nano-antigen protein (as shown in FIG. 4). The plasmid was transfected into HEK293F cells through a lipofection protocol, and a cell supernatant was harvested by centrifugation 3 days after transfection (the immunofluorescence image of 293F cells transfected with RBD-HP_Ferritin fusion protein is shown in FIG. 5), and a purification of target protein RBD-HP_Ferritin was carried out.

### 2. Purification of RBD-HP _Ferritin Nano-antigen

The supernatant of cells expressing RBD-HP_Ferritin was filtered through a µm filter to remove cell debris. After ultrafiltration through a 10 K ultrafiltration tube, the filtered cell supernatant was combined with Histrap-excel at 4° C. for 30 minutes, and a HisTrap excel nickel column was used for crude purification.

Afterwards, firstly 50 ml was washed with PBS (pH 7.4) buffer and low-concentration imidazole buffer (PBS, 50 mM Imidazole, pH 7.4) to remove flow-through impurity protein. Thereafter, target protein was eluted by high imidazole-containing buffer (PBS, 500 mM Imidazole, pH 7.4). Subsequently, the target protein was enriched using a Lectin Agarose column (GE) packed with Con A and WGA at a ratio of 1:1.

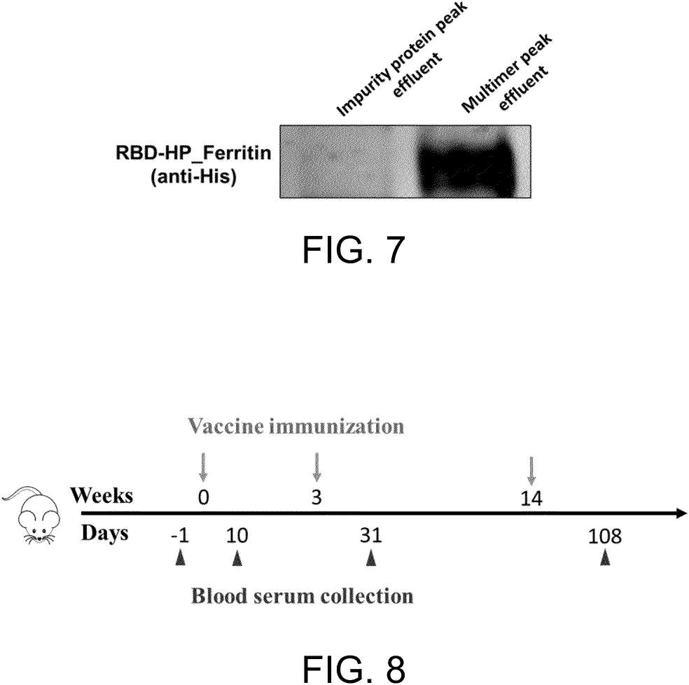

Elution peaks of RBD-HP_Ferritin 24-mer were collected and combined, and finally purified by molecular sieve chromatography using a Siperose6 Increase 10/300 GL column (GE) to obtain a 24-multimerized RBD-HP_Ferritin protein with a purity greater than 99% (as shown in FIGS. 6-7), a buffer for molecular sieve chromatography was: PBS, pH 7.4. After the target protein was concentrated, it was divided into small portions, quickly frozen in liquid nitrogen and stored at -80° C.

### Embodiment 2 Mouse Immunization Experiment

The RBD-HP _Ferritin antigen obtained in Embodiment 1 was diluted with physiological saline to 100 µg/ml according to Table 1, and emulsified in groups with an equal volume of adjuvant SAS. 6-8 week-old Balb/C mice were then immunized in groups. The immunization strategy was as shown in FIG. 8, that is, by intraperitoneal injection, each mouse received 3 times of vaccine immunization on Day 0, Week 3 (Day 21), and Week 14 (Day 108), with an inoculation volume of 200 µl (10 µg) each time. On Day 10, Day 31, and Day 108, the mice were bled from the orbit. Mouse blood serum was obtained by centrifugation at 4° C. and 2800 rpm for 15 minutes after standing for a period of time until the blood serum was precipitated, and was immediately used for SARS-CoV-2 pseudotyped viruspseudotyped virus neutralization detection experiment.

TABLE 1

| Antigen/control | Antigen content | Adjuvant | Number of animals |
|---|---|---|---|
| RBD-HP_Ferritin | 10 µg | SAS | 4 |
| PBS | 0 | SAS | 4 |

### Embodiment 3 Pseudotyped Virus Neutralization Test

### 1. Preparation of Pseudotyped Virus

According to a sequence published by NCBI, Spike protein of SARS-CoV-2 was synthesized and inserted into a pcDNA3.1 expression vector. 293T cells were co-transfected by the expression vector of SARS-CoV-2 Spike protein with pHIV-luciferase and psPAX2 plasmid. After 5 hours of transfection, cells were washed twice with PBS, and then continued to culture with replaced serum-free DMEM medium. After 48 hours, a supernatant was collected and centrifuged to remove cell debris. After dissolving with a small volume of serum-free DMEM, HIV-luc/SARS-CoV-2-S pseudotyped virus was obtained.

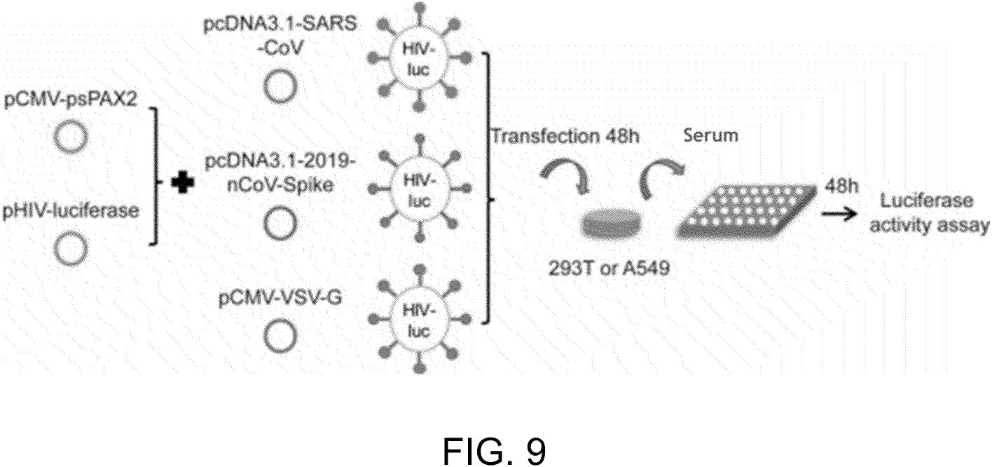

The pseudotyped virus can effectively simulate a process of wild-type SARS-CoV-2 invading cells. When it infects production cells or target cells, expression of luciferase reporter gene carried by SARS-CoV-2 pseudotyped virus can accurately reflect results of virus infection, so that results of the experimental system can be read accurately and quickly, which can be used as an excellent antibody neutralization titer monitoring system (as shown in FIG. 9).

### 2. Pseudotyped Virus TCID 50 Assay

The virus solution collected in the previous step was diluted 5-fold and added to HEK293T cells in a 96-well plate. After 4 hours of infection, the virus solution was discarded, cells were washed twice with PBS, replaced with DMEM complete medium containing 10% serum. After 48 hours, the medium was discarded, washed twice with PBS, added with a cell lysis buffer, and lysed by shaking for 30 minutes. After freeze-thawing once at -80° C., 30 µl of each well was taken to detect a luciferase activity value using GloMax 96 (Promega). TCID 50 was calculated by Reed-Muech method.

### 3. Neutralization Test

The purified antibody was diluted 2-fold, mixed with pseudotyped virus of TCID 50 final concentration, and co-incubated at 37° C. for 1 hour. The mixture was added to HEK293T cells with a density of about 70% in a 96-well plate. After 48 hours, culture medium was discarded, cells were washed twice with PBS, cell lysis buffer was added, and the luciferase activity value was detected.

### 4. Result Analysis

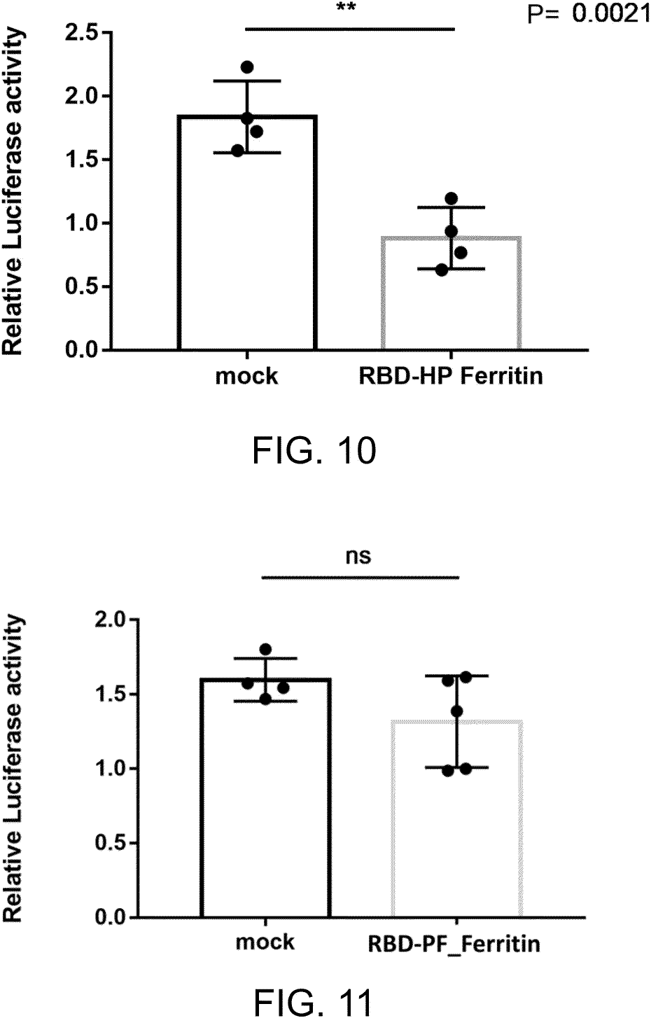

Results are shown in FIG. 10. A neutralizing activity against SARS-CoV-2 pseudotyped virus was detected in serum of Balb/c mice 10 days after immunization of RBD-HP_Ferritin nano-antigen. The t-test shows that there is a significant difference between an experimental group and a control group. At a significance level of 0.05, a two-tailed probability level is less than 0.05.

The results show that combination of RBD-HP_Ferritin of the present invention and SAS adjuvant can stimulate humoral immunity of mice 10 days after once immunization, which is less than neutralizing antibody titer stimulated by 24-mer group as a parallel control, and there is a significant difference.

### Comparative Example 1

Referring to the method of Embodiment 1, Helicobacter pylori multimeric protein (HP_Ferritin)was replaced by Pyrococcus furiosus multimeric protein (PF_Ferritin), and a fusion protein RBD-PF_Ferritin was constructed as an antigen.

And according to the method of Embodiments 2-3, mouse immunization experiment and pseudotyped virus neutralization test were carried out.

Results show (FIG. 11) that there was no significant difference in ability of serum of mice immunized with RBD-PF_Ferritin as an antigen to neutralize the SARS-CoV-2 pseudotyped virus compared to the control group.

### Comparative Example 2

Referring to the method of Embodiment 1, Helicobacter pylori multimer protein (HP_Ferritin)was replaced with dioxotetrahydropyridine synthase multimer protein (Lumazine Synthase, LS) derived from Aquifex aeolicus strain to construct a fusion protein LS- RBD as an antigen.

And according to the method of Embodiments 2-3, mouse immunization experiment and pseudotyped virus neutralization test were carried out.

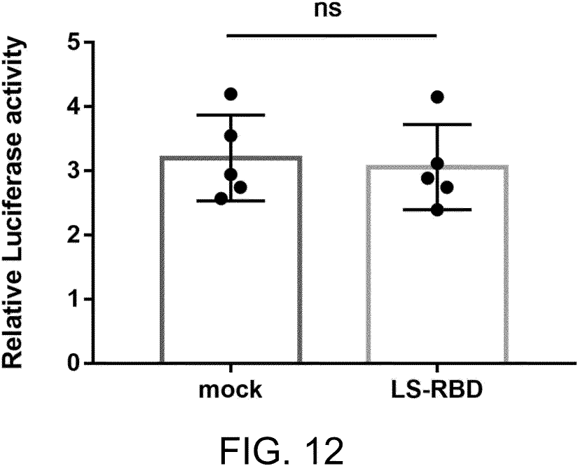

Results show (FIG. 12) that there was no significant difference in ability of serum of mice immunized with LS-RBD as an antigen to neutralize the SARS-CoV-2 pseudotyped virus compared to the control group.

The above-mentioned embodiments are preferred embodiments of the present invention, but the embodiments of the present invention are not limited by the above-mentioned embodiments, and any other changes, modifications, substitutions, combinations, and simplifications shall be equivalent replacement modes, which are all included in the protection scope of the present invention.

1. A method for improving antigen immunogenicity, comprising combining a receptor binding domain (RBD) of a virus with Helicobacter pylori multimeric protein (Helicobacter pylori_Ferritin, Ferritin (HP)) to form a new fusion protein RBD-HP_Ferritin, which is then used as an antigen.

2. The method according to claim 1,
wherein the antigen is a coronavirus antigen, and the receptor binding domain RBD of the virus are a receptor binding domain RBD of a coronavirus.

3. The method according to claim 2,
wherein the coronavirus antigen is a novel coronavirus SARS-CoV-2 antigen, and the receptor binding domain RBD of the coronavirus are a receptor binding domain RBD of a novel coronavirus SARS-CoV-2.

4. The method according to claim 3,
wherein the novel coronavirus SARS-CoV-2 antigen is a novel coronavirus SARS-CoV-2 surface spike protein (S protein) antigen.

5. The method according to claim 4,
wherein a sequence of the receptor binding domain RBD of the novel coronavirus SARS-CoV-2 is shown in SEQ ID NO: 1, an amino acid sequence of the Ferritin(HP) is shown in SEQ ID NO: 2, SEQ ID NO: 1 and SEQ ID NO: 2 can be directly linked, or the two can be linked by a hinge region Linker to form a new fusion protein RBD-HP_Ferritin; preferably, when the Linker is GSG, an amino acid sequence of the resulting fusion protein RBD-HP_Ferritin is shown in SEQ ID NO: 3.

6. The method according to claim 1,
wherein after the fusion protein is added with a signal peptide and a purification tag, an eukaryotic expression system is utilized to express antigen; preferably, the signal peptide is a secretory signal peptide (SP); preferably, the purification tag is a His tag (His-tag); preferably, an amino acid sequence of fusion of the SP, the His-tag, the receptor binding domain RBD of the novel coronavirus SARS-CoV-2 and HP_Ferritin is as shown in SEQ ID NO: 4.

7. A coronavirus antigen, wherein a fusion protein RBD-HP_Ferritin is constructed and obtained according to the method in claim 1.

8. Use of the coronavirus antigen in claim 7 in preparation of anti-coronavirus medicament.

9. The use according to claim 8, wherein the use is to combine the coronavirus antigen and a SAS adjuvant.

10. The use according to claim 8, wherein the use is for preparation of a kit; the kit contains the antigen, or a DNA molecule encoding the antigen, or a recombinant vector/ expression kit/ transgenic cell line/recombinant bacterium expressing the antigen.

11. A recombinant vector, expression kit, transgenic cell line or recombinant bacterium expressing the coronavirus antigen of claim 7.

12. A coronavirus vaccine, wherein the coronavirus vaccine is prepared by the coronavirus antigen of claim 7 as an antigen.

13. A preparation method of the antigen of claim 7, comprising: at a 3′ end of a nucleotide sequence corresponding to amino acids as shown in direct linking or hinge linking of SEQ ID NO: 1 and SEQ ID NO: 2, or a nucleotide sequence corresponding to amino acids as shown in SEQ ID NO: 3 or SEQ ID NO: 4, adding a translation terminator codon, performing clone expression, screening for a correct recombinant, then transfecting an eukaryotic expression system for expression, collecting a cell supernatant after expression, and purifying to obtain the novel coronavirus antigen.

14. A nucleotide sequence encoding and expressing the coronavirus antigen of claim 7, or a vector or transgenic cell line comprising the sequence.

15. A coronavirus antigen, wherein a fusion protein RBD-HP_Ferritin is constructed and obtained according to the method in claim 3.

16. A coronavirus antigen, wherein a fusion protein RBD-HP_Ferritin is constructed and obtained according to the method in claim 5.

17. A coronavirus antigen, wherein a fusion protein RBD-HP_Ferritin is constructed and obtained according to the method in claim 6.

18. A coronavirus vaccine, wherein the coronavirus vaccine is prepared by the coronavirus antigen of claim 15 as an antigen.

19. A coronavirus vaccine, wherein the coronavirus vaccine is prepared by the coronavirus antigen of claim 16 as an antigen.

20. A coronavirus vaccine, wherein the coronavirus vaccine is prepared by the coronavirus antigen of claim 17 as an antigen.
